(19) United States
(12) Patent Application Publication
(10) Pub. No.: US 2024/0000788 A1
(43) Pub. Date: Jan. 4, 2024

(54) USE OF PENTOXIFYLLINE IN PREPARATION OF MEDICAMENT FOR REPAIRING ENDOTHELIAL GLYCOCALYX DAMAGE

(71) Applicant: Air Force Medical University, Xi'an City (CN)

(72) Inventors: Xiao-di Zhang, Xi'an City (CN); Chen-qian Zhao, Xi'an City (CN); Jiang-zheng Liu, Xi'an City (CN); Meng-meng Liu, Xi'an City (CN); Rui Liu, Xi'an City (CN); Chun-xu Hai, Xi'an City (CN)

(21) Appl. No.: 18/201,441

(22) Filed: May 24, 2023

(30) Foreign Application Priority Data
Jun. 29, 2022 (CN) 2022107558193

(51) Int. Cl. A61K 31/522; A61P 39/02

(52) U.S. Cl. CPC A61K 31/522; A61P 39/02

(57) ABSTRACT

Use of pentoxifylline (PTX) in preparation of a medicament for repairing endothelial glycocalyx damage, and belongs to the technical field of medicine. A chlorine-induced endothelial glycocalyx damage model is established. After the endothelial glycocalyx damage model is successfully established, the PTX is intraperitoneally injected into a rat with endothelial glycocalyx damage. The result shows that the PTX effectively repairs shedding of glycosaminoglycans and syndecans in the endothelial glycocalyx damage and significantly downregulates the expression of inflammatory factors including tumor necrosis factor alpha (TNF-α), interleukin-6 (IL-6), and matrix metallopeptidase 13 (MMP-13). Therefore, the PTX can effectively repair the endothelial glycocalyx damage, providing a new theoretical basis for repairing the endothelial glycocalyx damage. Furthermore, with high safety, the PTX has the prospect of being developed into the medicament for repairing the endothelial glycocalyx damage.

### CROSS REFERENCE TO RELATED APPLICATION

This patent application claims the benefit and priority of Chinese Patent Application No. 2022107558193, filed with the China National Intellectual Property Administration on Jun. 29, 2022, the disclosure of which is incorporated by reference herein in its entirety as part of the present application.

### TECHNICAL FIELD

The present disclosure belongs to the technical field of medicine, in particular to use of pentoxifylline in preparation of a medicament for repairing endothelial glycocalyx damage.

### BACKGROUND

As a chemical raw material, chlorine gas is widely used in papermaking, textile and other industrial production. It is also widely used in hospitals, swimming pools, tap water disinfection and pharmaceutical industry. Chlorine gas (Cl₂) is an asphyxiant. While it is widely used, its accidental leakage has caused huge threats and losses to people's life and property security, and directly affects normal production, living order, and social security.

Endothelial glycocalyx (EG) is the most luminal layer of the blood vessel, growing on and within the vascular wall, with a thickness of 0.5-10.0 mm, mainly composed of proteoglycans, glycosaminoglycans (GAGs), side chains, glycoproteins, and some plasma proteins. The most prominent components of proteoglycans are mainly syndecans. The most common GAGs include heparan sulfate (HS), chondroitin sulfate (CS), and hyaluronan (HA). Among them, HS has the highest content, accounting for 50% to 90%. The core protein skeleton mainly contains syndecans, glypicans, and perlecans, which construct an important structural basis for EG to exert its main physiological functions.

At present, it is believed that EG is an important regulator of microcirculation function. EG plays important roles in maintaining the stability of endothelial cell structure and function, inhibiting microthrombosis, regulating the microcirculation blood flow and the roles of blood cells and endothelial cells, preventing the adhesion of inflammatory cells, and maintaining the integrity of the vascular wall barrier function. Exposure of endothelial cells to mechanical forces induced by blood flow, particularly shear stress, determines the morphology and function of the endothelial cell. Endothelial cells exposed to shear stress produce NO, which is an important determinant of vascular tone. NO released by vascular endothelial cells can inhibit platelet aggregation and leukocyte adhesion to the vascular wall, the hyperplasia of the vascular smooth muscle, and the infiltration of mononuclear macrophages. Thus, disruption of the glycocalyx impairs a plurality of important endothelial cell functions, reduces NO secretion, and leads to impaired mechanotransduction, including changes in fluid shear stress, activation of coagulation pathways, leukocyte and platelet adhesion to endothelial cell surfaces, leakage of fluid and plasma proteins into the interstitium, and the resulting tissue edema due to vascular barrier dysfunction.

However, vascular endothelial injury caused by chlorine poisoning is related to the increase of vascular permeability. Therefore, drugs that can be used to repair endothelial glycocalyx damage are urgently needed to repair endothelial glycocalyx damage and ensure vascular health.

### SUMMARY

In view of this, an objective of the present disclosure is to provide use of pentoxifylline in preparation of a medicament for repairing endothelial glycocalyx damage.

To achieve the above objective, the present disclosure provides the following technical solution:

The present disclosure provides use of pentoxifylline in preparation of a medicament for repairing endothelial glycocalyx damage.

Preferably, the endothelial glycocalyx damage may include chlorine-induced endothelial glycocalyx damage.

Preferably, the medicament may include active pharmaceutical ingredient pentoxifylline and a pharmaceutically acceptable carrier.

Preferably, the medicament may effectively repair shedding of glycosaminoglycans and syndecans in the endothelial glycocalyx damage.

Preferably, the medicament may significantly downregulate the expression of an inflammatory factor.

Preferably, the inflammatory factor may include one or more selected from the group consisting of tumor necrosis factor alpha (TNF-α), interleukin-6 (IL-6), and matrix metallopeptidase 13 (MMP-13).

Compared with the prior art, the present disclosure has the following beneficial effects:

The present disclosure provides use of pentoxifylline in preparation of a medicament for repairing endothelial glycocalyx damage. A chlorine-induced endothelial glycocalyx damage model is established. After the endothelial glycocalyx damage model is successfully established. the pentoxifylline (PTX) is intraperitoneally injected into a rat with endothelial glycocalyx damage. The result shows that the PTX effectively repairs shedding of glycosaminoglycans and syndecans in the endothelial glycocalyx damage and significantly downregulates the expression of inflammatory factors including TNF-α, IL-6, and MMP-13. Therefore, the PTX can effectively repair the endothelial glycocalyx damage, providing a new theoretical basis for repairing the endothelial glycocalyx damage. Furthermore, with high safety, the PTX has the prospect of being developed into the medicament for repairing the endothelial glycocalyx damage.

### BRIEF DESCRIPTION OF THE DRAWINGS

FIG. 1 shows fluorescence staining results of main components (CS, GAG, and syndecan-1) of endothelial glycocalyx in lung tissues in different treatment groups;

FIGS. 2A, 2B, and 2C show expression levels of TNF-α, IL-6, and MMP-13 in the sera of different treatment groups detected by enzyme-linked immunosorbent assay (ELISA); and

FIGS. 3A, 3B, and 3C show results of the content of main components (CS, GAG, and syndecan-1) of endothelial glycocalyx in bronchoalveolar lavage fluids (BALFs) of different treatment groups detected by ELISA.

### DETAILED DESCRIPTION OF THE EMBODIMENTS

The present disclosure studies endothelial glycocalyx damage, and finds that PTX can effectively repair the endothelial glycocalyx damage, especially chlorine-induced endothelial glycocalyx damage. Therefore, use of PTX in preparation of a medicament for repairing endothelial glycocalyx damage is provided.

In the present disclosure, the PTX belongs to a derivative of methylxanthine. The molecular formula thereof is C₁₃H₂₀N₄O₃, having the following chemical structure:

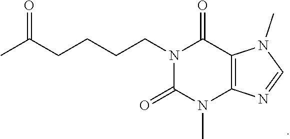 [SMILES: CC(=O)CCCCn1c(=O)c2c(ncn2C)n(C)c1=O]

In the present disclosure, the endothelial glycocalyx damage may preferably include chlorine-induced endothelial glycocalyx damage. If a concentration-time product of the chlorine is 400 ppm×15 min, the chlorine-induced endothelial glycocalyx damage may significantly cause significant shedding and decomposition of GAGs and syndecans.

In the present disclosure, the medicament may preferably include active pharmaceutical ingredient PTX and a pharmaceutically acceptable carrier. In the present disclosure, the carrier may be one selected from the group consisting of a diluent, an antioxidant, a disintegrant, an excipient, and a wetting agent, including one or more selected from the group consisting of starch, dextrin, calcium sulfate, lactose, mannitol, sucrose, sodium chloride, glucose, syrup, honey, glucose solution, mucilago acaciae, gelatin mucilage, sodium carboxymethylcellulose, polyoxyethylene sorbitan fatty acid esters, sodium dodecyl sulfonate, methylcellulose, ethylcellulose, corn starch, stearates, boric acid, liquid paraffin, and polyethylene glycol (PEG). The medicament may include dosage forms formulated in the form of a pulvis, a granule, a tablet, a capsule, a dropping pill, a pill, a powder, a lyophilized powder for injection, a solution, a suspension, an emulsion, or a film. Routes of administration of the medicament in the present disclosure may be selected from the group consisting of oral administration, intranasal administration, intravenous injection, intramuscular injection, subcutaneous injection, intraperitoneal injection, and intradermal injection. A daily dosage of the medicament in the present disclosure may be 0.001-150 mg/kg body weight, preferably 0.01-100 mg/kg body weight, and more preferably 0.01-60 mg/kg body weight.

A chlorine-induced endothelial glycocalyx damage model is established in the present disclosure. After the model is successfully established, the PTX is intraperitoneally injected into a rat with endothelial glycocalyx damage. The result shows that the PTX effectively repairs shedding of GAGs and syndecans in the endothelial glycocalyx damage and significantly downregulates the expression of inflammatory factors including TNF-α, IL-6, and MMP-13. Therefore, the PTX can effectively repair the endothelial glycocalyx damage.

The technical solution provided by the present disclosure will be described in detail below with reference to the examples, but they should not be construed as limiting the claimed scope of the present disclosure.

### Example 1

Laboratory animals: 24 male SD (200 t 20 g) rats were purchased from the Animal Center of the Air Force Medical University (Xi'an, China), and the laboratory animal license number was SCXK (Shaanxi) 2019-001. Six animals were housed in each cage and raised in a clean barrier animal feeding system provided by the Laboratory Animal Center of the Air Force Medical University. The drinking water was tap water, the bedding material was sawdust, the ambient temperature was (24±2)° C., and the relative humidity was 50%±10%. The animals were maintained on a 12 h:12 h light/dark cycle, and fasted but not deprived of water 12 h before exposure.

1. Establishment of the chlorine-induced endothelial glycocalyx damage model: The rats were placed in a systemic exposure device for static exposure. The concentration-time product of chlorine was 400 ppm×15 min. After 15 min, the rats were taken out and exposed for 3 h to obtain rat models of chlorine-induced endothelial glycocalyx damage.

2. The protective effect of PTX on endothelial glycocalyx after chlorine exposure:

Experimental grouping: The rats were randomly divided into a control group (Control), a chlorine exposure group (endothelial glycocalyx damage model group, Cl₂), and a PTX intervention group (Cl₂+PTX), 8 rats in each group.

(1) The PTX intervention group was treated as follows: the rats were placed in the systemic exposure device for static exposure; the concentration-time product of chlorine was 400 ppm×15 min; after 15 min, the rats were taken out, immediately intraperitoneally injected with 50 mg/kg PTX in normal saline, and sacrificed after 3 h. The chlorine exposure group was treated as follows: the rats were placed in the systemic exposure device for static exposure; the concentration-time product of chlorine was 400 ppm×15 min; after 15 min, the rats were taken out, immediately intraperitoneally injected with the same volume of normal saline as the PTX intervention group, and sacrificed after 3 h. The rats in the control group were placed in a clean systemic exposure device without chlorine under the same conditions, taken out after 15 min, and immediately intraperitoneally injected with the same volume of normal saline as the PTX intervention group, and sacrificed after 3 h; and

(2) Sera, BALFs, and the remaining lung tissues were collected from the rats in the control group (Control), the chlorine exposure group (endothelial glycocalyx damage model group, Cl₂), and the PTX intervention group (Cl₂+PTX), respectively.

Paraffin sections were used to detect CS, GAG and syndecan-1 in lung tissue across groups by immunofluorescence assay, and levels of HS, CS, syndecan-1, TNF-α, IL-6, and MMP 13 in serum and BALF across groups were detected by ELISA.

The steps of immunofluorescence assay on paraffin sections were as follows:
1) Deparaffinization of paraffin sections into water: The sections were successively in xylene I for 15 min, xylene II for 15 min, absolute ethanol I for 5 min, absolute ethanol I1 for 5 min, 85% alcohol for 5 min, and 75% alcohol for 5 min, and washed with distilled water.
2) Antigen retrieval: Tissue sections were placed in a retrieval box filled with citrate antigen retrieval buffer (pH 6.0) for antigen retrieval in a microwave oven, heated over moderate heat for 8 min until boiled, ceased for 8 min, and then switched to moderate-low heat to heat for 10 min. In this process, the buffer should be prevented from excessive evaporation, and the slides should not be dried. After natural cooling, the slides were placed in PBS (pH 7.4), shaken and washed thrice on a destaining shaker, for 5 min each time.

3) Circling and serum sealing: The sections were slightly spin-dried and circled around the tissue using a PAP Pen (to prevent the antibody from flowing away); the PBS was spin-dried, and 3% bovine serum albumin (BSA) was added dropwise to block for 30 min.

4) Addition of primary antibody: The blocking buffer was gently shaken off, the primary antibody prepared with PBS in a given proportion was added dropwise on a section, the section was laid flat in a wet box and incubated overnight at 4° C., and a small volume of water was added to the wet box to prevent the antibody from evaporating.

5) Addition of secondary antibody: The slides were placed in PBS (pH 7.4), shaken and washed thrice on the destaining shaker, for 5 min each time. After each section was slightly spin-dried, the secondary antibody corresponding to the primary antibody of the species was added dropwise into the circle to cover the tissue and incubated in the dark at room temperature for 50 min.

6) 4′,6-Diamidino-2-phenylindole (DAPI) counterstaining of cell nuclei: The slides were placed in PBS (pH 7.4), shaken and washed thrice on the destaining shaker, for 5 min each time. After each section was slightly spin-dried, DAPI staining solution was added dropwise into the circle and incubated in the dark at room temperature for 10 min.

7) Quenching of tissue autofluorescence: The slides were placed in PBS (pH 7.4), shaken and washed thrice on the destaining shaker, for 5 min each time, and an autofluorescence quencher was added to the circles for 5 min and washed with running water for 10 min.

8) Mounting: The sections were slightly spin-dried and mounted with Antifade Mounting Medium.

9) Microscopy and photography: The sections were observed under a fluorescence microscope and images were acquired. The ultraviolet excitation wavelength of DAPI was 330-380 nm, its emission wavelength was 420 nm, and blue light was emitted; the excitation wavelength of FITC was 465-495 nm, its emission wavelength was 515-555 nm, and green light was emitted; the excitation wavelength of CY3 was 510-560 nm, its emission wavelength was 590 nm, and red light was emitted.

ELISA:

An anesthetized rat was immobilized in the supine position. The thoracic cavity of the rat was opened, and the blood was drawn from the abdominal aorta. The right hilum was ligated, the cervical trachea was separated, a catheter was inserted, and BALF was collected by repeated irrigation with 2.5 mL of normal saline for thrice. The BALF was centrifuged at 1,500 r/min for 10 min at 4° C., and the supernatant was collected to detect the levels of HS, CS, and syndecan-1 in BALF and levels of TNF-α, IL-6, and MMP-13 in serum in accordance with the instructions of the ELISA kit.

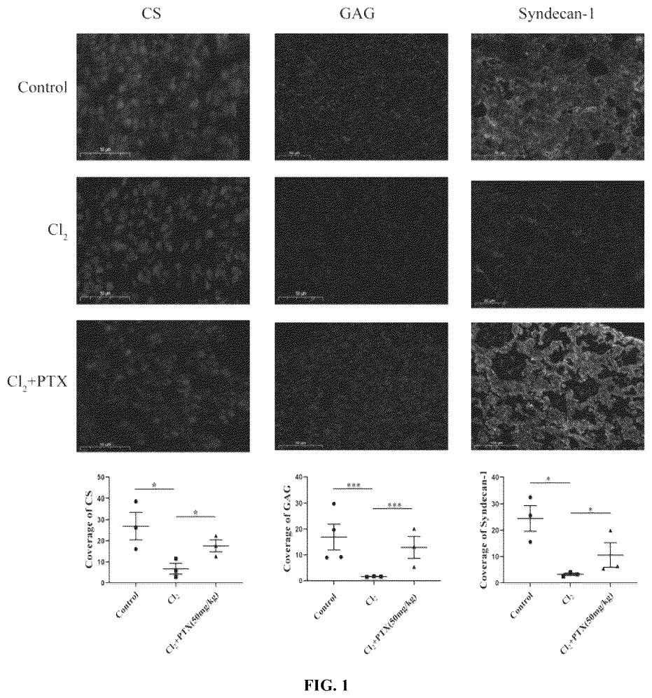

According to the results, compared with the control group, the main components (CS, GAG, and syndecan-1) of the endothelial glycocalyx in the lung tissue of the chlorine exposure group were obvious shed and decomposed, the levels of TNF-α, IL-6, and MMP-13 in serum rose significantly, and the main components (CS, GAG, and syndecan-1) of the endothelial glycocalyx in BALF increased significantly. These indicated that the chlorine-induced endothelial glycocalyx damage model was successfully established (see FIGS. 1 to 3).

The results in FIG. 1 show that the shedding of the main components (CS, GAG, and syndecan-1) of the endothelial glycocalyx in the chlorine-induced lung tissue is effectively repaired in the PTX intervention group compared with the chlorine exposure group, indicating that the intervention is effective. * P<0.05, **P<0.01, ***P<0.0001; n=8, scar bar=50 μm, 100 μm.

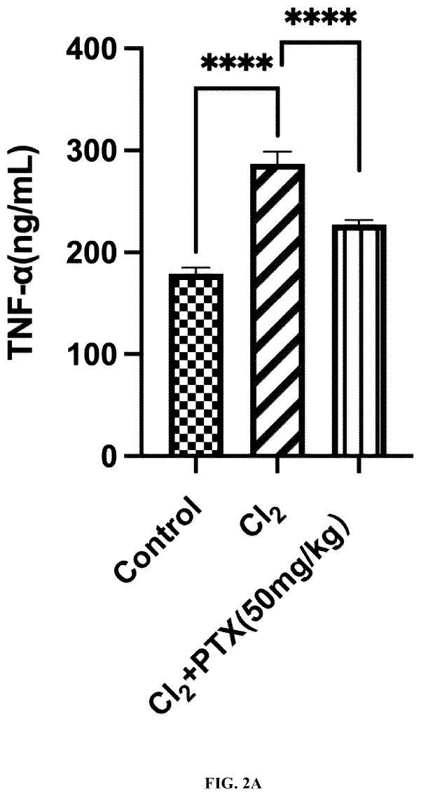

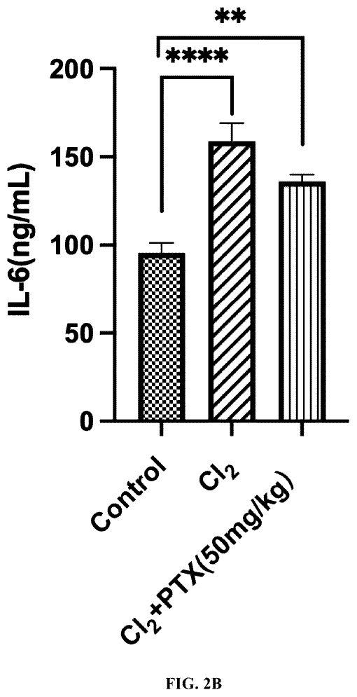

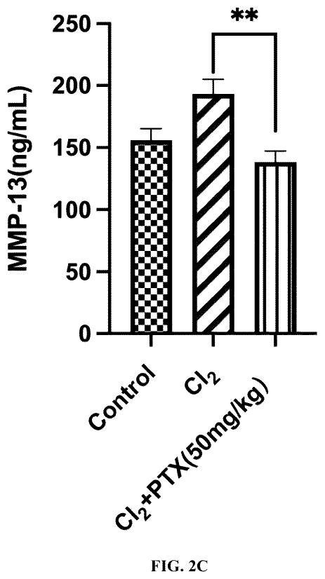

The results in FIGS. 2A-C show that the PTX intervention group exhibits a significant improvement in serum TNF-α, IL-6, and MMP-13 compared with the chlorine exposure group, indicating that the intervention is effective. * P<0.05, **P<0.01. ***P<0.0001; n=8.

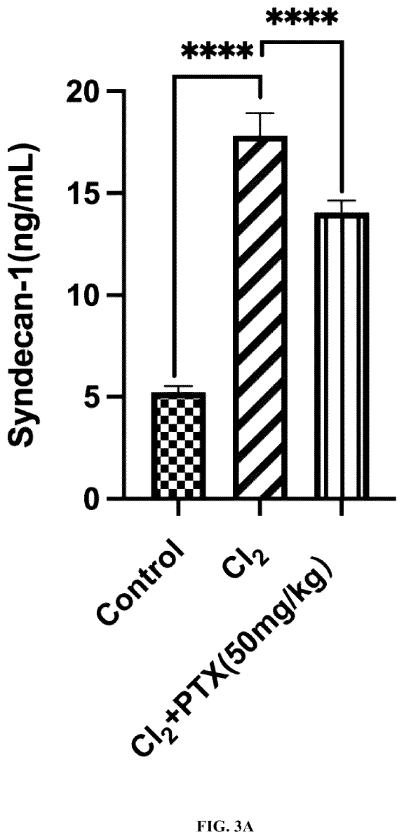

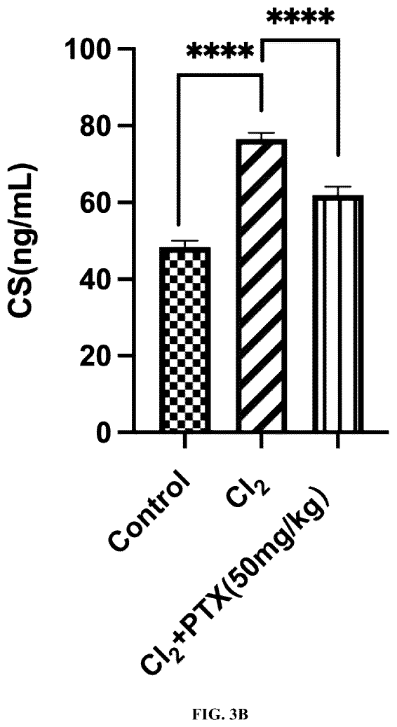

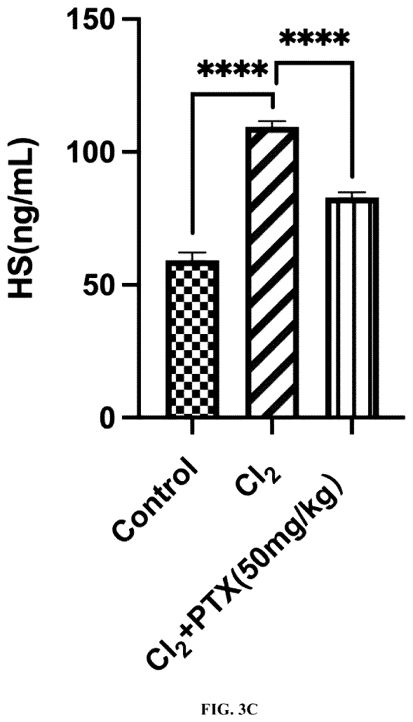

The results in FIGS. 3A-C show that the PTX intervention group exhibits significant decreases in the main components (CS, GAG, and syndecan-1) of the endothelial glycocalyx in the BALF compared with the chlorine exposure group, indicating that PTX (50 mg/kg) effectively repairs the chlorine-induced shedding of the main components (CS, GAG, and syndecan-1) of the endothelial glycocalyx, namely that the intervention is effective, * P<0.05, **P<0.01, ***P<0.0001; n=8.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A medicament for repairing endothelial glycocalyx damage, comprising pentoxifylline.

2. The medicament for repairing endothelial glycocalyx damage according to claim 1, Wherein the endothelial glycocalyx damage comprises chlorine-induced endothelial glycocalyx damage.

3. The medicament for repairing endothelial glycocalyx damage according to claim 1, wherein the medicament comprises active pharmaceutical ingredient pentoxifylline and a pharmaceutically acceptable carrier.

4. The medicament for repairing endothelial glycocalyx damage according to claim 1, wherein the medicament effectively repairs shedding of glycosaminoglycans and syndecans in the endothelial glycocalyx damage.

5. The medicament for repairing endothelial glycocalyx damage according to claim 1, Wherein the medicament significantly downregulates expression of an inflammatory factor.

6. The medicament for repairing endothelial glycocalyx damage according to claim 5, wherein the inflammatory factor comprises one or more selected from the group consisting of tumor necrosis factor alpha (TNF-α), interleukin-6 (IL-6), and matrix metallopeptidase 13 (MMI′-13).
